(19) United States
(12) Patent Application Publication
(10) Pub. No.: US 2024/0408314 A1
(43) Pub. Date: Dec. 12, 2024

(54) STABILIZER FOR MEDICATION DELIVERY DEVICE

(71) Applicant: Eli Lilly and Company, Indianapolis, IN (US)

(72) Inventor: Jaime Ray ARNETT, Fishers, IN (US)

(21) Appl. No.: 18/699,114

(22) PCT Filed: Oct. 4, 2022
(86) PCT No.: PCT/US2022/045594

§ 371 Date: Apr. 5, 2024

Related U.S. Application Data
(60) Provisional application No. 63/253,754, filed on Oct. 8, 2021.

(51) Int. Cl. A61M 5/315; A61M 5/20

(52) U.S. Cl. CPC A61M 5/31548; A61M 5/20; A61M 2005/2073; A61M 2205/3576

(57) ABSTRACT

The present disclosure relates to a medication delivery device including a stabilizing apparatus for effective delivery of a dose of medication without interference with various device components or misapplication of pressure to the device by a user. The stabilizing apparatus couples to the medication delivery device and facilitates proper force delivery to the underlaying medication delivery device.

### FIELD OF THE DISCLOSURE

The present disclosure relates to medication delivery devices and related methods of use. Specifically, the present disclosure relates to medication delivery devices having a guard or guide to facilitate medication delivery using a medication delivery device.

### BACKGROUND OF THE DISCLOSURE

Patients suffering from various diseases must frequently inject themselves with medication. To allow a person to conveniently and accurately self-administer medicine, a variety of devices broadly known as pen injectors or injection pens are available. Generally, these pens are equipped with a cartridge including a piston and containing a multi-dose quantity of flowable medication, including liquids. A drive member is movable to advance the piston in the cartridge to dispense the contained medication from an outlet at the distal cartridge end, typically through a needle. Control of the movement of the piston controls the amount of medication delivered by the medication delivery device.

Many pen injectors and other medication delivery devices utilize mechanical systems in which members rotate and/or translate relative to one another in a manner proportional to the dose delivered by operation of the device. Interference with movement of these members may interfere with delivery of medication or otherwise cause confusion for patients as to whether the proper dosage of medication has or has not been delivered. Additionally, users with dexterity impairment may be unable to effectively and easily use such devices.

### SUMMARY OF THE DISCLOSURE

In exemplary embodiments of the present disclosure includes medication delivery devices which may include a stabilizing apparatus for effective delivery of a predetermined dose of medication without interference with various device components or misapplication of pressure to the device by a user. The exemplary stabilizing apparatus may couple to the medication delivery device and may facilitate proper force delivery to the underlaying medication delivery device.

In a first aspect, an apparatus for a medication delivery device is disclosed. The medication delivery device having a body, and a user dose setter coupled to a proximal end of the body. The user dose setter including a proximal surface facing away from a needle end of the body and a circumferential surface extending from the proximal surface and defined about a longitudinal axis of the medication delivery device. The apparatus including a guide body associated with the medication delivery device, a slide button axially movable relative to the guide body, and at least one rail coupled between the guide body and the slide button. The slide button includes a shielding surface and a shielding lip. The shielding surface at least partially covers the proximal surface of the user dose setter of the medication delivery device. The shielding lip extends toward the needle end along the circumferential surface of the user dose setter of the medication delivery device. During dose setting and delivery, the slide button is rotationally free relative to the user dose setter and axially movable relative to the guide body. During dose setting, the user dose setter is rotated in a first direction, the user dose setter together with the slide button proximally move away from the body of the medication delivery device from a first position to a second position.

In another aspect, disclosed is a medication delivery system including a medication delivery device disposed about a longitudinal axis and having an actuator, and a data collection device couplable to the actuator and rotationally and axially fixed relative to the actuator. The data collection device includes an input element that is axially movable relative to the data collection device and is configured to activate the actuator for dose delivery. A guide body is associated with the medication delivery device. A slide button is axially movable relative to the guide body and rotationally free relative to the data collection device. The slide button includes an attachment element configured for removable attachment to a portion of the data collection device. A first rail and a second rail are coupled between the guide body and the slide button. The slide button includes a shielding surface at least partially covering a proximal surface of the data collection device. The first and second rails and the shielding surface are arranged to provide a window to define a human graspable section of the data collection device for rotation of the data collection device for dose setting of the medication delivery device.

In another aspect, an assembly for a medication delivery device is disclosed. The assembly includes a guide body having at least one receiver, at least one rail positionable within the at least one receiver, a slide button coupled to the at least one rail opposite the at least one receiver, and a data collection device positioned intermediate of the slide button and the guide body, the data collection device configured to facilitate coupling of the stabilizing apparatus to the medication delivery device.

In another aspect, disclosed is a method of removably attaching a stabilizing apparatus to a medication delivery device, the method including or more of the following steps: providing an apparatus comprising a guide body having at least one receiver, a slide button having at least one rail positioned within the at least one receiver, and a data collection device coupled to the slide button such that the data collection device is positioned intermediate of the slide button and the guide body; providing a medication delivery device; inserting the medication delivery device within a through hole defined by the guide body; and attaching an actuator of the medication delivery device to the data collection device.

Additional features and advantages of the present invention will become apparent to those skilled in the art upon consideration of the following detailed description of the illustrative embodiment exemplifying the best mode of carrying out the invention as presently perceived.

### BRIEF DESCRIPTION OF THE DRAWINGS

The foregoing aspects and many additional features of the present invention and accompanying methods will become more readily appreciated and become better understood by reference to the following detailed description when taken in conjunction with the accompanying figures. The detailed description of the drawings particularly refers to the accompanying figures in which:

FIG. 1 illustrates an exemplary embodiment of a medical delivery device, including a body, an actuator, and a needle;

FIG. 2 illustrates an exemplary stabilizing apparatus coupled to the medical delivery device of FIG. 1, the stabilizing apparatus including a guide body with at least one receiver and a rail received within the receiver and coupled to a slide button, the slide button covering a portion of the actuator of the medical delivery device;

FIG. 3 is a top view that illustrates coverage of the proximal side of the actuator of the medical delivery device of FIG. 1 by the slide button of the stabilizing apparatus of FIG. 2;

FIG. 4 is an exploded view of the stabilizing apparatus and medical delivery device of FIG. 2;

FIG. 5 is a longitudinal cross section of the at least one receiver and the rail of the stabilizing device of FIG. 2, illustrating the interaction of the rail with a limit element;

FIG. 6 is a partial longitudinal cross-section of an embodiment of the slide button, wherein the slide button couples to a portion of an embodiment of the data collection device such as, for example, in FIG. 3A;

FIGS. 7A-C are illustrations of the coupling of an embodiment of the stabilizing apparatus of FIG. 6 with the medical delivery device of FIG. 1;

FIG. 8 is a diagrammatic illustration of an embodiment of the stabilizing apparatus coupled to the medical delivery device of FIG. 1;

FIG. 9 is a perspective view of an aspect of the stabilizing apparatus of FIG. 8 coupled to the medical delivery device of FIG. 1 with a data collection device;

FIG. 10 is an illustration of an embodiment of the stabilizing apparatus with the medical delivery device of FIG. 1;

FIG. 11A is an illustration of a first exemplary grip that a user may use to effectively utilize the medical delivery device and any of the embodiments of the stabilizing apparatus as disclosed herein;

FIG. 11B is an illustration of a second exemplary grip that a user may use to effectively utilize the medical delivery device and any of the embodiments of the stabilizing apparatus as disclosed herein;

FIG. 12 is an illustration of a third exemplary grip that a user may use to effectively utilize the medical delivery device and any of the embodiments of the stabilizing apparatus as disclosed herein;

FIG. 13 is a perspective view of an embodiment of the stabilizing apparatus with a medical delivery device;

FIG. 14 is a perspective view of an embodiment of the stabilizing apparatus with a medical delivery device having a data collection device coupled thereto;

FIG. 15 is a perspective view of another embodiment of the slide button of the stabilizing apparatus; and

FIG. 16 is a perspective view of another embodiment of the slide button of the stabilizing apparatus.

FIG. 17 is a cross-sectional area view of another embodiment of the stabilizing apparatus.

Corresponding reference characters indicate corresponding parts throughout the several views. Although the drawings represent embodiments of various features and components according to the present disclosure, the drawings are not necessarily to scale, and certain figures may be exaggerated in order to better illustrate and explain the present disclosure.

### DETAILED DESCRIPTION OF THE DRAWINGS

For the purposes of promoting an understanding of the principles of the present disclosure, reference will now be made to the embodiments illustrated in the drawings, which are described herein. The embodiments disclosed herein are not intended to be exhaustive or to limit the invention to the precise form disclosed. Rather, the embodiments are chosen and described so that others skilled in the art may utilize their teachings. Therefore, no limitation of the scope of the claimed invention is thereby intended. The present invention includes any alterations and further modifications of the illustrated devices and described methods and further applications of principles in the invention which would normally occur to one skilled in the art to which the invention relates.

The terms “couples”, “coupled”, “coupler” and variations thereof are used to include both arrangements wherein the two or more components are in direct physical contact and arrangements wherein the two or more components are not in direct contact with each other (e.g., the components are “coupled” via at least a third component), but yet still cooperate or interact with each other.

In some instances, throughout this disclosure and in the claims, numeric terminology, such as first, second, third, fourth, etc., is used in reference to various components or features. Such use is not intended to denote an ordering of the components or features. Rather, numeric terminology is used to assist the reader in identifying the components or features being referenced and should not be narrowly interpreted as providing a specific order of components or features.

Embodiments of a stabilizing apparatus are disclosed herein that may be useful in the dose setting and/or dose delivery with a medication delivery device. The stabilizing apparatus may be coupled to an actuator of a medication delivery device or to a data collection device that is coupled to an actuator of a medication delivery device. One of the many benefits of such a stabilizing apparatus is to allow control movement of the actuator or data collection device during the outward spiraling of the dose setting member of a medication delivery device during dose setting. Another of the many benefits of such a stabilizing apparatus is to help control movement of the actuator or data collection device during the downward spiraling of the dose setting member of a medication delivery device during dose delivery. Another of the many benefits of such stabilizing apparatus is to allow the user to access the means for rotating during dose setting, such as, e.g., the actuator or the data collection device. Because the stabilizing apparatus is usable for medication delivery devices without and with a data collection device, the term “user dose setter” may be used to refer to both the actuator of the medication delivery device when a data collection device is not present or to the data collection device when present and coupled over the actuator.

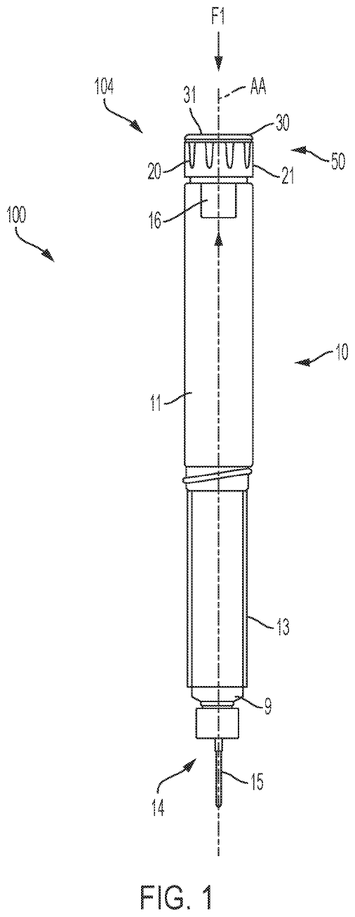

Referring initially to FIG. 1 an exemplary medication delivery device 100 is illustrated. Medication delivery device 100 (hereinafter “device”) includes an elongated pen-shaped body 10, including a distal portion 13 and a proximal portion 11. In some embodiments, distal portion 13 may define a cartridge holder which may include a reservoir or cartridge 9 configured to hold a medication to be dispensed through an outlet 14 during a dispensing operation. The term “medication” refers to one or more therapeutic agents including, but not limited to epinephrine, anesthetics, analgesics, steroids, insulins, insulin analogs such as insulin lispro or insulin glargine, insulin derivatives, GLP-1 receptor agonists such as dulaglutide or liraglutide, glucagon, glucagon analogs, glucagon derivatives, gastric inhibitory polypeptide (GIP), GIP analogs, GIP derivatives, combined GIP/GLP-1 agonists such as tirzepatide, basal insulins, oxyntomodulin analogs, oxyntomodulin derivatives, therapeutic antibodies including but not limited to IL-23 antibody analogs or derivatives, such as mirikizumab, IL-17 antibody analogs or derivatives, such as ixekizumab, therapeutic agents for pain-related treatments, such as galcanzeumab or lasmiditan, therapeutic agents for atopic dermatitis, such as lebrikizumab and any therapeutic agent that is capable of delivery by the devices described herein. Medication delivery devices according to the present disclosure are operated in a manner generally as described herein by a user (for example, a healthcare professional, a caregiver, or another person) to deliver one or more medications to a patient (for example, another person or the user).

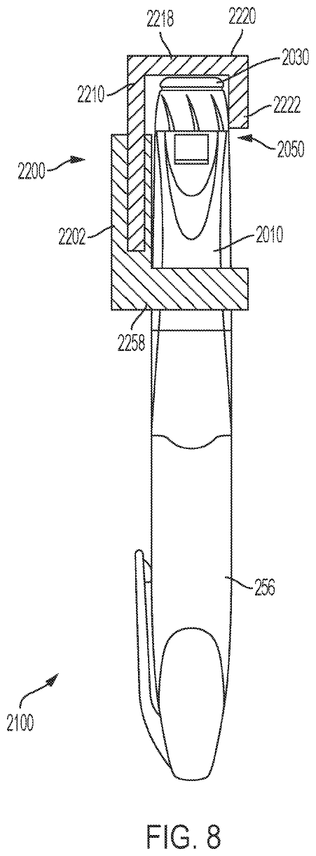

Outlet 14 of distal portion 13 may be equipped with an injection needle 15. In some embodiments, injection needle 15 may be removable from a needle hub of the distal portion 13 of body 10. In some embodiments, injection needle 15 may be replaced with a new injection needle after each use. In other embodiments, body 10 may be reusable, and the cartridge may be configured to be replaced. Device 100 may also include a pen cap 256 (shown in FIG. 8) to cover or otherwise protect the cartridge holder and needle hub of the distal portion 13 of body 10 after injection needle 15 has been removed from the distal portion 13 of body 10.

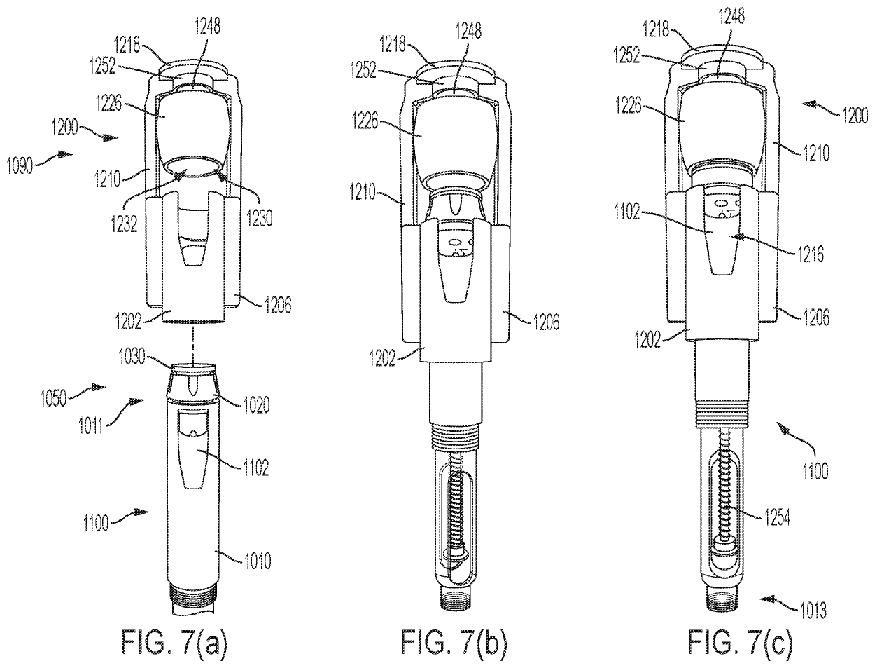

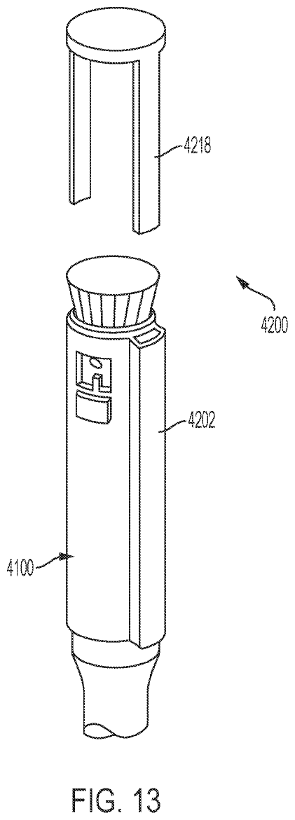

Proximal portion 11 of body 10 may include a drive member (shown as drive member 1254 in FIG. 7C), which may include a screw or another suitable driving mechanism, configured to transfer force from a user applied to an actuator 50 to a piston located in distal portion 13 to deliver a predetermined dose of medication out of needle 15. Although drive member 1254 is illustrated in connection with the embodiment FIG. 7C, any embodiment described herein may include such drive member 1254. Accordingly, drive member 1254 may be axially moveable relative to body 10 along axis AA, wherein axis AA extends longitudinally relative to body 10. Device 100 may include a rotatable dose selection member 20, e.g., a collar, and an activation button 30 positioned at a proximal end 104 of body 10. Rotatable member 20 and activation button 30 may form an actuator 50 as described further herein. For example, in some embodiments, rotatable member 20 and the activation button 30 may be two separate components which act in cooperation as actuator 50. In other embodiments, rotatable member 20 and activation button 30 may be formed by single-piece construction to form actuator 50 so that rotatable member 20 and activation button 30 are not two discrete components. Such an embodiment may be, for example, found in KwikPen™ provided by Eli Lilly and Company (Indianapolis, Indiana) and is shown in FIG. 13. Coupled between the actuator 50 and the drive member 1254 may be a dose setting member 17 that may be one or more rotatable dose setting members, such as a dose dial with numbers, sleeves, other members threadably engaged, that can rotate and/or translate during dose setting and dose dispensing and interface with the drive member 1254 to move the piston. While a single rotatable member 20 is illustrated herein, some embodiments may include more than one rotatable member 20. Activation button 30 may be mechanically coupled to the drive member of proximal portion 11 such that depression of activation button 30 may result in ejection of the medication as previously described. Activation button 30 may include a push surface, or proximal surface 31, to facilitate application of a distally directed force F1 to activation button 30 to operate device 100. Activation button 30 may be attached to device 100 by being directly positioned on, received within, integral with, or otherwise connected to a component of device 100. Connections may include, for example, frictional engagement, splines, a snap or press fit, sonic welding, or adhesive. In other words, actuator 50 may be mechanically coupled to the drive member of proximal portion 11 such that depression of actuator 50 may result in ejection of the medication as previously described. Proximal surface 31 faces proximally away from the needle end of body 10 and is configured to facilitate application of a distally directed force F1 to actuator 50 to operate device 100.

Device 100 may be operable in a dose setting mode. For example, rotatable member 20 may include a circumferential surface 21 extending away from the activation button 30. The circumferential surface 21 is defined and positioned about longitudinal axis AA of the device 100 and is configured to facilitate rotation of the rotatable member 20 in one of a clockwise or counterclockwise direction to adjust and select the dosage (e.g., volume of medication to be injected).

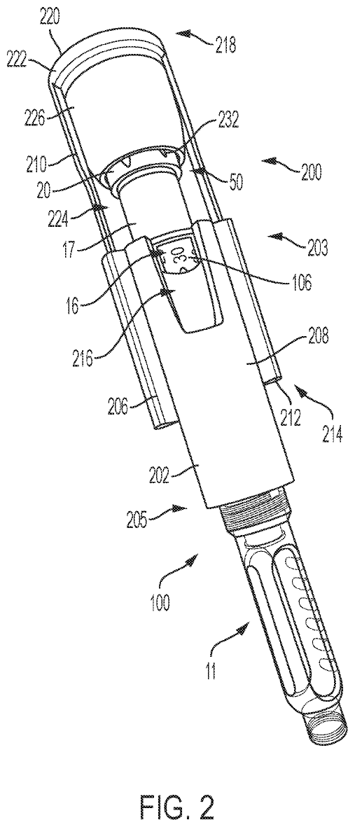

Rotatable member 20 and activation button 30 may be rotatably fixed to one another during the dose setting mode of operation, thereby allowing the unscrewing of the dose setting member 17 away from a zero position during dose setting such that the rotatable member 20 and activation button 30, i.e., the actuator 50, rotates and axially travels in the proximal direction relative to the pen body 10 (such as to the position shown in FIG. 2). The zero position is defined when the dose setting member is fully screwed inside relative to the pen body 10 and the actuator 50 is in its closest proximity to the pen body 10, as shown in FIG. 1. In other words, rotation of rotatable member 20 may also cause activation button 30 to rotate. In other embodiments, rotatable member 20 and activation button 30 may be rotationally independent during the dose setting mode of operation. Device 100 may additionally or alternatively be operable in a dose dispensing mode, in which activation button 30 is axially translated along axis AA in response to a user pressing on activation button 30. Dose dispensing and dose delivery may be used herein interchangeably. During dose dispensing, activation button 30 may be freely rotatable relative to body 10 when depressed in some embodiments, while in other embodiments activation button 30 may be rotatably fixed relative to body 10 when depressed. During dose dispensing, rotatable member 20 rotates in the direction opposite from the dose setting direction discussed above, thereby allowing the screwing of the dose setting members to return to the zero position such that the rotatable member 20 rotates and axially travels in the distal direction relative to the pen body 10. In other words, rotatable member 20 may be rotatable about longitudinal axis AA relative to body 10 in a first direction during dose setting and may be rotatable about longitudinal axis AA relative to body 10 in a second direction during dose dispensing, where the first direction may be opposite of the second direction.

Activation button 30 may be axially translatable relative to rotatable member 20, which may be separated from activation button 30 by a gap. Axially translating activation button 30 toward rotatable member 20 to reduce the gap may trigger the dose dispensing mode. In some embodiments, rotatable member 20 may rotate as activation button 30 is axially translated toward rotatable member 20. In some embodiments, rotatable member 20 and activation button 30 may become rotationally uncoupled in the dose dispensing mode, such that rotatable member 20 rotates relative to activation button 30 during dispensing of fluid.

Rotating rotatable member 20 in the first direction may serve to increase the predetermined dose and rotating rotatable member 20 in the second direction may serve to decrease the predetermined dose during the dose setting mode. Rotatable member 20 may be rotationally adjustable in pre-defined rotational increments corresponding to a minimum incremental increase or decrease of the set dose during the dose setting operation. Rotatable member 20 may include a detent mechanism such that each rotational increment produces an audible and/or tactile “click.” For example, one increment or “click” may equal one-half or one unit of the medication. In some embodiments, the predetermined dose amount may be visible via series of dial indicator markings 106 (FIG. 2) shown through a dosage window 16.

Once the predetermined dose of medication is set by rotating rotatable member 20, device 100 may be manipulated so that injection needle 15 properly penetrates, for example, a user's skin. The dose dispensing mode of operation may be initiated in response to an axial distal force F1 applied to proximal surface 31 of activation button 30, which may interact with a drive member of device 100 to deliver the medication to the user. The dose dispensing mode of operation may be completed when actuator 50 has returned to its zero-dose position. Rotatable member 20 may rotate relative to body 10 while activation button 30 is rotationally stationary relative to body 10 during dose dispensing mode, during which rotatable member 20 and activation button 30 travel together in the distal direction.

In some embodiments, device 100 may further include a data collection device or a sensing module (referenced herein a data collection device 226, 1226, 2226, 5226) coupled to or incorporated with the rotatable member 20. The data collection device may be rotationally fixed relative to the rotatable member 20 such that at least a portion of the data collection device is rotatable about longitudinal axis AA relative to the body 10 during dose setting and may be rotatable about longitudinal axis AA relative to body 10 during dose dispensing. Data collection device may be configured to allow axial movement of one part of the data collection device relative to another part, and in this manner, initial pressing of the slide button allows the force to transmit to the activation button via the one part and the data collection device body. Data collection device may determine information that may correspond to the amount of dose set and/or dose delivered, the time of delivery, a kind of pen/drug product, etc. The determination of dose set and/or dose delivered may be based on relative rotation between at least a portion of data collection device and body 10 and/or based on relative rotation between two portions of data collection device in some embodiments. Data collection device may include a controller to process and communicate output signals from one or more sensors of data collection device representative of the sensed relative rotation. Data collection device may include an electronics assembly suitable for operation of the data collection device as described herein. The controller is operably connected to the data collection device to receive outputs from one or more rotational sensors. The controller may include conventional components such as a printed circuit board, a processor, power supply, memory, microcontrollers, etc. contained for example in the body of data collection device. Alternatively, at least some components may be provided separately, such as by means of a computer, smart phone, or other device. Means are then provided to operably connect the external controller components with the sensor arrangement at appropriate times, such as by a wired or wireless connection, e.g., Bluetooth®, Wi-Fi, cellular, NFC, or other wireless means.

For sensing relative rotation, the data collection device housing may include a first part that rotates relative to a second part. One of the parts is associated with a sensed component, and the other of the parts is associated with a sensing component. Sensing of relative rotation may occur during dose setting and/or dose delivery. In one embodiment, the housing of the data collection device may include two housing components that rotate together during dose setting and that rotate relative to one another during dose delivery. One example of this arrangement is described in U.S. Patent Publication Number 2021/0330891, filed Jan. 11, 2019, and entitled, “Dose Detection Module For a Medication Delivery Device,” which is hereby incorporated by reference in its entirety. In another example, the housing of the data collection device may be counted as the first part, and an axially movable button on top of the device may constitute the second part. One example of this arrangement is described in PCT Patent Application Number PCT/2022/35402, filed Jun. 29, 2022, and entitled, “Medication Delivery Device with Dose Button,” which is hereby incorporated by reference in its entirety. In another example, the upper housing of the data collection device may be counted as the first part, and a lower housing of the data collection device may constitute the second part, whereby the upper housing is movable axially relative to the lower housing and upon axial movement the activation button is depressed for dose delivery to initiate.

During a dose setting mode of operation, the data collection device may be coupled to rotatable member 20 and activation button 30 such that rotation of the data collection device by the user causes rotation of the rotatable member 20 relative to body 10 along longitudinal axis AA of device 100. A user may rotate at least a portion of data collection device relative to the housing to set a dose. Data collection device may include a portion that is axially movable relative to the body of the data collection device to engage the activation button 30 such that movement of the portion causes depression of the activation button 30 to initiate dose dispensing. In the dose dispensing mode, at least a portion of the data collection device may be rotatable relative to body 10 in concert with the rotating rotatable member 20 about longitudinal axis AA of the device 100.

It should be appreciated that while a distinct body and arrangement is shown for device 100, rotatable member 20, and activation button 30, such description as provided herein may apply across several embodiments with differing shapes and arrangements as can be found, for example, in Ergo II provided by Eli Lilly and Company (Indianapolis, Indiana) and/or KwikPen™ provided by Eli Lilly and Company (Indianapolis, Indiana).

Further details of the design and operation of exemplary embodiments of a delivery device 100 may be found in U.S. Pat. No. 7,195,616, entitled “Medication Injector Apparatus with Drive Assembly that Facilitates Reset”; and U.S. Pat. No. 7,291,132 entitled “Medication Dispensing Apparatus with Triple Screw Threads for Mechanical Advantage”, each of which are hereby incorporated by reference in their entireties. For example, one embodiment of the dose setting members 17 is described in the '132 patent.

It should be understood that the various systems, components, features, and methods described relative to these embodiments may be used either individually and/or in any desired combination as the disclosure is not limited to only the specific embodiments described herein. For example, while device 100 has been described in the form of a pen injector, device 100 may be any device which is used to set and deliver a predetermined dose of a medication, such as pen injectors, autoinjectors, bolus injectors, and syringes. The medication may be any one of a type that may be delivered by such a device. The device may be a reusable device capable of receiving a replaceable and/or disposable cartridge of medication or may be an entirely disposable device with a prefilled reservoir of medication.

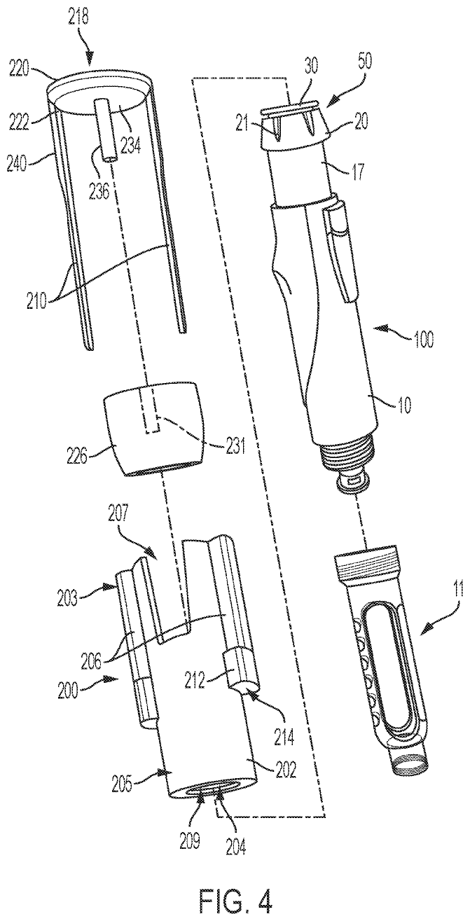

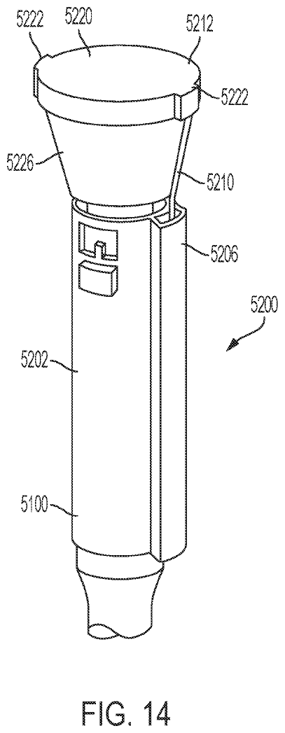

Now referring to FIG. 2 and FIG. 4, device 100 including an embodiment of a stabilizing apparatus 200 is illustrated. Stabilizing apparatus 200 includes a guide body 202 configured to couple to body 10 of device 100 and a slide button 218 slidably coupled with the guide body 202. The slide button 218 may be coupled with the guide body 202 via a rail 210 as discussed further herein. In one embodiment, rail 210 may be directly coupled to device 100. For example, rail 210 may be directly coupled with device 100 and translate axially with respect to device 100 during dose setting and/or dose delivery. Device 100 may further provide a better coupling relationship between the rail 210 and corresponding guide body receiver 206 to prevent unwanted movement of rail 210 within corresponding receiver 206. For example, a sidewall 208 may not be coupled between rail 210 and device 100. In other embodiments, rail 210 may not be directly coupled to device 100, but instead be coupled to device 100 via guide body 202 (FIG. 14) wherein rail 210 may translate axially to device 100. For example, sidewall 208 may be coupled between rail 210 and device 100.

Guide body 202 is generally hollow, e.g., forms through hole 204 (FIG. 4), and may be shaped corresponding to an outer perimeter of body 10 so that guide body may receive body 10 to couple to body 10 via friction fit. Guide body 202 may define any shape that facilitates coupling of guide body 202 to body 10 of device 100. In some embodiments, guide body 202 may include a protruding feature that couples the guide body 202 to body 10 of device 100 or another feature of device 100 such as the dosage window 16. In other embodiments, guide body 202 may also receive a bezel or other protruding feature of the body 10 of the device 100 where such arrangement may facilitate attachment and/or angular orientation of the guide body 202 with the device body 10. Guide body 202 may also otherwise couple to body 10 via splines, a snap or press fit, sonic welding, adhesive, mechanical fasteners, or a combination thereof. Guide body 202 may be removably coupled to the body 10 of the device 100 or permanently coupled to or form integrally with the body 10 of the device 100. In the illustrated embodiment, through hole 204 is fully enclosed by the material of the guide body 202. In other embodiments, the through hole 204 may be partially enclosed by the material. For example, in some embodiments, guide body may have a C-shaped cross-sectional shape. In some embodiments where the guide body has a C-shaped cross-sectional shape, the guide body 202 may include a longitudinal gap along guide body 202, wherein the longitudinal gap of guide body 202 may spread apart to receive device body 10. As shown, through hole 204 extends through a proximal end 203 of guide body 202 and a distal end 205 of guide body 202 to define proximal end opening 207 (FIG. 4) and distal end opening 209 (FIG. 4), respectively, so that device body 10 is received by through hole 204. For example, device body 10 may extend beyond proximal end opening 207 and also extend beyond distal end opening 209 of guide body 202, which such embodiment is shown in FIG. 7B.

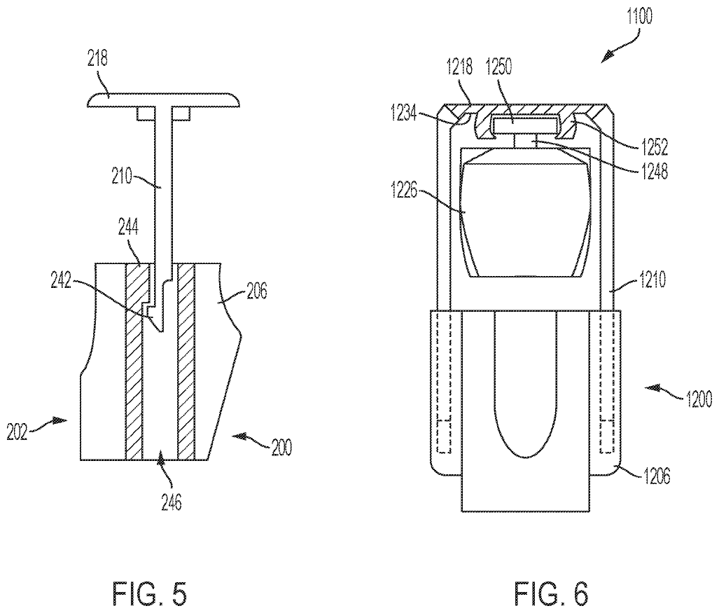

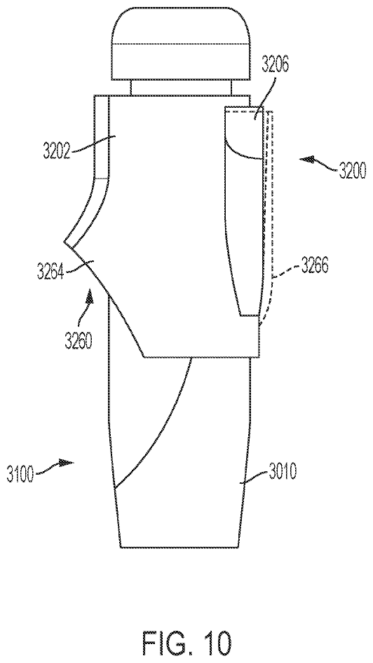

Guide body 202 includes at least one receiver 206 positioned on and extending axially from sidewall 208 of the guide body 202. The receiver 206 is configured to receive a rail 210 within a corresponding pocket 246 (FIG. 5) of receiver 206, wherein rail 210 is coupled to slide button 218 as described further herein. As illustrated in FIG. 2, guide body 202 may include two receivers 206, wherein each receiver 206 receives a corresponding rail 210 as described further herein. In other embodiments, guide body 202 may include a greater number of receivers 206, including, for example, three, four, five, or six receivers 206, wherein each receiver 206 receives a corresponding rail 210. Each rail 210 is circumferentially spaced from adjacent rail(s) 210 to define at least one window 224 (shown in FIG. 2). For example, in an embodiment including two rails 210, two opposite-facing windows 224 would be defined between the rails 210. Window 224 is sized and shaped to allow access to the actuator 50 so that actuator 50 may be directly rotated during dose setting and/or dose dispensing. In one embodiment, the actuator 50 may be rotated and the stabilizing apparatus is not rotated. As described above, receivers 206 may extend axially outward from guide body 202 so that a lower surface 212 of receiver 206 forms a ledge 214 to facilitate grip of a user as described further herein. Ledge 214 may be formed extending radially outwardly away from axis from the guide body 202 and may be formed at least partially or fully around the circumference of device 100. In some embodiments where at least two receivers 206 exist, a user may use ledges 214 to facilitate a grip on device 100 so that the user may supply a dose from device 100 using a technique similar to the use of a syringe. In other embodiments, a single ledge 214 may be formed radially outwardly away from axis of guide body 202 where a user may use the single ledge to facilitate using a technique similar to the use of a syringe. In some embodiments, a separate ledge 214 may be provided on guide body 202 spaced apart from receivers 206. Guide body 202 may further define at least one slot 216 to expose dosage window 16, including potential dial indicator markings 106 as discussed above. Slot 216 may also receive a bezel or other protruding feature of the body 10 of the device 100 where such arrangement may facilitate attachment and/or angular orientation of the guide body 202 with the device body 10, also shown in FIG. 10. Slot 216 may be disposed circumferentially spaced from the receiver 206 and may be in fluid communication with the proximal end opening 207. In some embodiments, guide body 202 may cover dosage window 16 covering slot 216. In such embodiments, guide body 202 should be made of a transparent material, e.g., polycarbonate, to view dosage window 16.

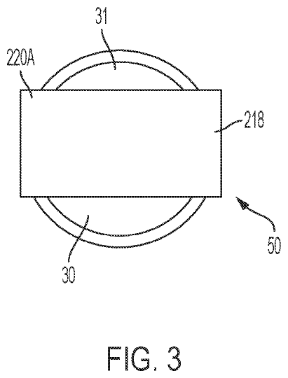

Slide button 218 includes a shielding surface 220, which is configured to at least partially cover proximal surface 31 (FIG. 1) of the actuator 50 or the proximally facing surface of data collection device upon assembly. For example, referring additionally to FIG. 3, shielding surface 220A may cover at least 40%, at least 50%, at least 60%, at least 70%, at least 80%, at least 90%, at least 95% of the proximal surface 31 of actuator 50 as shown or the proximal surface of the data collection device. Such coverage allows for a large target for users, while providing the further benefits of the stabilizing apparatuses described herein. Several embodiments described herein, such as the one in FIG. 2, show the shielding surface 220 covering the entirety of the proximal surface of the data collection devices described herein or the proximal surface of actuator if data collection device was not used. Referring again to FIG. 2, upon assembly, the slide button 218 may be axially locked relative to the actuator 50 while axially moveable relative to the guide body 202. Further, slide button 218 may contact actuator 50, but not interfere with the movement of actuator 50 such as the rotation of actuator 50, in some embodiments. Slide button 218 further includes a shielding lip 222, which extends laterally beyond a circumference of actuator 50 or data collection devices described herein and extends proximally toward the proximal portion of the respective device.

In the illustrated embodiment in FIG. 2, data collection device 226 may be coupled to the actuator 50 and disposed between the slide button 218 and the guide body 202. Data collection device 226 may form a friction fit or be rotationally fixed with actuator 50 so that actuation of data collection device 226 correspondingly actuates actuator 50; for example, rotation of data collection device 226 may correspondingly rotate actuator 50 for operation of device 100. As described later with reference to FIG. 6, an interior surface of data collection device 226 that define a cavity may include coupling features to couple with the rotatable member. For example, the coupling features may include one of a plurality of protrusions or recessions configured to mate with the other of protrusions or recessions of rotatable member 20 (FIG. 2) (shown as projections 21 in FIG. 4) to facilitate operable connection between data collection device 226 and actuator 50.

Referring now to FIG. 4, an exploded view of device 100 and corresponding stabilizing apparatus 200 is provided. As shown, slide button 218 includes a distal surface 234. A protrusion 236 may be extending from the distal surface 234, wherein protrusion 236 is configured to transmit an axial force, such as force F1 discussed in relation to FIG. 1, to actuator 50 so that, during dose delivery, actuator 50 and slide button 218 move, in tandem, toward body 10 of device 100 to a first position in which medication is delivered. When actuator 50 is rotated in the first direction, as described above, to set a subsequent medication dose, actuator 50 provides force to slide button 218 so that actuator 50 and slide button 218 move, in tandem, away from body 10 of device 100 from the first position to a second position, at which actuator 50 and slide button 218 is primed for delivery of the set medication dose. Protrusion 236 is received within a through hole 231 of data collection device 226 (shown by dashed lines) to contact activation button 30 of actuator 50 when force is applied to the shielding surface 220 during dose delivery. Protrusion 236 may be configured to facilitate coupling of the data collection device to the slide button. Protrusion 236 may be configured to facilitate rotation of the data collection device relative to the slide button.

Each of rails 210 may include a rib 240 so that each of rails 210 includes a T-shaped cross-section to provide extra stability and strength to each corresponding rail 210. The rib 240 may further provide a better coupling relationship between each rail 210 and corresponding guide body receiver 206 to prevent unwanted movement of rail 210 within corresponding receiver 206. Each rail 210 may also include a limit element 242 as shown in FIG. 5, which is configured to engage with a lip 244 within pocket 246 of receiver 206. Limit element 242 may be a circumferential tab that extends beyond the rail body. As stabilizing apparatus 200 moves from the first position to the second position during dose setting, limit element 242 engages lip 244 to prohibit further proximal movement of rail 210 and slide button 218 during dose setting beyond a predefined axial distance as defined by the position of lip 244. The axial length of each rail 210 provides each rail 210 with a range of translational motion sufficient to couple guide body 202 to slide button 218 via rail 210 when device 100 is fully extended in the second position and fully depress activation button 30 (FIG. 1) to the first position to deliver a maximum dose and/or after administering a complete dose.

Now referring to FIG. 6, medication delivery device 1100 and another embodiment of stabilizing apparatus 1200 as described below are substantially the same as device 100 and stabilizing apparatus 200 described above, except as further described herein, wherein like components are like-numbered with the addition of “1000” added to the original reference number.

In another embodiment, a portion of data collection device 1226 includes an input element operable coupled to the actuator 1050, such as, for example, when pressed engages the activation button 1030 to initiate dose delivery. Input element is shown having a stem 1248, which is coupled to, for example, the proximal axial surface of the data collection device 1226. Stem 1248 extends in a distal direction within the body of the data collection device 1226 toward the actuator 1050 of the device 1100 to engage, for example, the activation button 1030 during dose delivery. Tab 1250 may be connected to the proximal side of the stem 1248 and include a larger cross-sectional area than the body of the stem 1248. Slide button 1218 includes an attachment element 1252 extending from distal surface 1234. Attachment element may form a cylindrical wall. In one example, attachment element 1252 is configured to engage tab 1250. In other embodiments, the attachment element 1252 may be sized and shaped to engage the proximal end of the data collection device alone, or in addition to the tab if included. Data collection device 1226 can interact directly with activation button 1030 during operation of device 1100. In other embodiments, attachment element 1252 may engage directly with activation button 30 (FIG. 1) so that actuation of slide button 1218 directly actuates activation button 30 during operation of device 1100. As illustrated in FIG. 6, attachment element 1252 may be a snap-type element that snaps onto tab 1250 (as shown) or the proximal end of the data collection device. For example, attachment element 1252 may include a cylindrical wall extending about axis AA from the distal surface 1234 of slide button 1218 so that an inner surface of the cylindrical wall engages and is removably locked to the radially outer surface of tab 1250, preferably via an interference fit. In other embodiments, attachment element 1252 may include two or more arms extending distally from distal surface 1234 of slide button 1218 to radially engage tab 1250 via interference fit or snap-type fit. In other embodiments, other attachment methods may be used including an attachment element 1252 such as that illustrated in FIG. 7, wherein attachment element 1252 defines a circular opening to receive stem 1248. Attachment elements described herein are rotationally free and axially fixed relative to the data collection device or actuator during operation of the device.

FIGS. 7A-C illustrate the assembly 1090 of device 1100 and stabilizing apparatus 1200, similar to the assembly of device 100 and stabilizing apparatus 200 described above. Attachment element 1252 is shown coupled to data collection device 1226 by receiving tab 1250 of stem 1248 of data collection device 1226 as described above in relation to FIG. 6. In FIG. 7A, guide body 1202 receives the proximal portion 1011 of body 1010 of device 1100 to couple to body 1010 via friction fit or any other attachment method as described above in relation to body 10 and guide body 202. In another embodiment, guide body 1202 receives the distal portion 1013 of body 1010 of device 1100 so that slot 1216 of guide body 1202 receives the bezel 1102 of device 1100 which can aid in orienting and/or fixing the guide body 1202 axially and rotationally relative to device 1100. Guide body 1202 includes at least one receiver 1206 with a pocket sized and shaped to receive rail 1210 as described above. With additional reference to FIG. 7B, data collection device 1226 receives actuator 1050, including activation button 1030 and rotatable member 1020 so that actuation of data collection device 1226 also actuates actuator 50 as described above. Specifically, data collection device 1226 includes a distal end opening 1230 leading to an internal cavity 1232 sized and shaped to receive actuator 1050. As described, the interior surface defining the cavity 1232 includes coupling features for coupling with the rotatable member 1030. In FIG. 7C, the final assembly is shown with the stabilizing apparatus 1200 coupled to the data collection device 1226 and to the device 1100. As with device 100 and stabilizing apparatus 200, stabilizing apparatus 1200 may not include data collection device 1226, so that slide button 1218 couples directly to activation button 30. As tab 1250 is axially pressed in relative to the body of the data collection device 1226 by the slide button 1218 to start injection, a distal end of the tab internal to the data collection device engages the activation button 30 of the device. During dose delivery, the tab 1250 does not rotate relative to the data collection device 1226 thereby giving the relative rotation for sensing rotational position and/or rotational movement that corresponds to dose delivered by the electronics assembly.

Now referring to FIG. 8, device 2100 is shown coupled to another embodiment of stabilizing apparatus 2200 as described below are substantially the same as device 100 and stabilizing apparatus 200 described above, except as further described herein, wherein like components are like-numbered with the addition of “2000” added to the original reference number. This is to also show that any of the stabilizing apparatuses described herein can be attached directly to an actuator of a medication delivery device that does not have a data collection device.

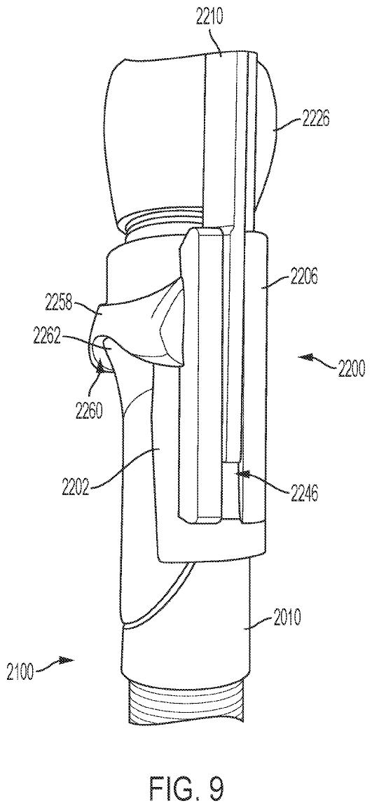

Stabilizing apparatus 2200 includes a guide body 2202 which extends down a single side of body 2010. Stabilizing apparatus 2200 includes a coupling ring 2258 that is configured to receive body 2010 of the medication delivery device. Coupling ring 2258 may be a full ring-shape configured to couple with body 2010 via friction fit. In other embodiments, coupling ring 2258 may be a partial ring, which snaps onto body 2010. Other attachment methods may be used as described above in relation to stabilizing apparatus 200 and body 10. Guide body 2202 receives rail 2210, which extends to slide button 2218. Slide button 2218 couples or otherwise interacts with actuator 2050 as described above. For example, slide button 2218 may include shielding surface 2220 which may fully or partially cover activation button 2030 as described above in relation to activation button 30. Shielding lip 2222 extends away from shielding surface 2220 to couple with activation button 2030. For example, shielding lip 2222 may be an axial portion opposite rail 2210 that extends distally to at least partially cover the axial extent of the radial outer surface of the actuator and/or data collection device. In other embodiments, shielding lip 2222 may extend distally around a portion of or the entirety of the perimeter of slide button 2218. In other embodiments, slide button 2218 may couple to activation button 2030 via an attachment element as described above in relation to attachment element 1252 (FIG. 6), including a snap-type element or friction fit element. It is understood that such embodiment of stabilizing apparatus 2200 may be also applied to a medication delivery device having a data collection device as described herein, such as, for example, shown in FIG. 9.

FIG. 9 further illustrates the interaction of guide body 2202, rail 2210, and body 2010. As shown, rail 2210 is received within pocket 2246 of receiver 2206 of guide body 2202 so that rail 2210 and guide body 2202 operate similar to guide body 202 and rail 210 described above. Additionally, coupling ring 2258 may include a recess 2260, configured to receive a protrusion 2262 of body 2010 to facilitate attachment between guide body 2202 and body 2010 of device 2100. As shown, stabilizing apparatus 2200 may include a data collection device 2226 as described above.

Now referring to FIG. 10, in another embodiment, a device 3100 and a stabilizing apparatus 3200 as described below are substantially the same as device 100 and stabilizing apparatus 200 described above, except as further described herein, wherein like components are like-numbered with the addition of “3000” added to the original reference number.

Stabilizing apparatus 3200 includes guide body 3202 receiving body 3010 and including a radial protrusion 3264 to serve as a stop for axial movement of a user's hand toward the needle end of body 3010. Guide body 3202 wraps around body 3010 to facilitate coupling of guide body 3202 to body 3010. Guide body 3202 further includes a cover 3266 to cover the bezel of the device 3100. Cover 3266 may be formed of a clear material to allow viewing of the bezel and may further be formed of magnifying material to facilitate clear and accurate reading of the underlying dial indicator numbers 3206. Cover 3266 snaps over the bezel to prevent movement of cover 3266 relative to the bezel, which may further facilitate attachment of stabilizing apparatus 3200 to body 3010. Such cover 3266 may be included with any other stabilizing apparatus as described above, including stabilizing apparatus 200, 1200, 2200. Like guide body 2202, guide body 3202 may include a recess 3260, configured to receive a protrusion of body 3010 to facilitate attachment between guide body 3202 and body 3010 of device 3100.

After any one of the stabilizing apparatuses disclosed herein is operationally coupled to the device, a user may place the device in dose setting operation. With reference to FIG. 2, when stabilizing apparatus 200 is coupled to the device 100, the user can still access through the window 254 defined by the rail(s) 210 and rotate the actuator 50 or the data collection device 226 coupled to the actuator 50 in the dose setting direction. In some embodiments, rotation of the actuator 50 and/or data collection device in the dose setting direction causes unscrewing of the dose setting member 17 along with the actuator and/or data collection device to rotate and proximally move relative to guide body 202 coupled to device 100. In turn, the shielding surface 220 of slide button 218 travels proximally away from device 100 as the rail 210 slides further out of corresponding receiver 206 of the guide body 202. As such, the stabilizing apparatus provides the benefit of allowing the user to access and rotate the actuator and/or data collection device with minimal impact to the normal dose setting operation of the device. If a limit element is present with the stabilizing apparatus, the limit element may allow the user to set a fixed dose or a max dose with the device.

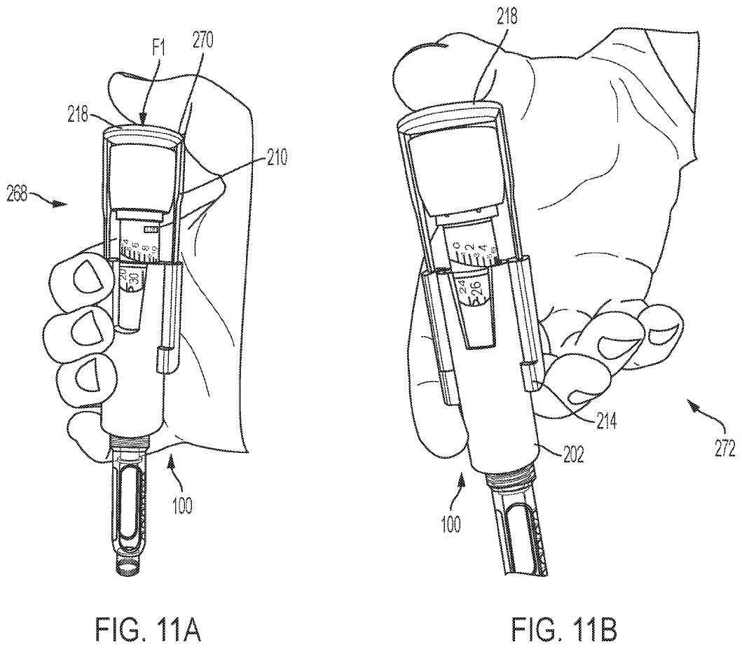

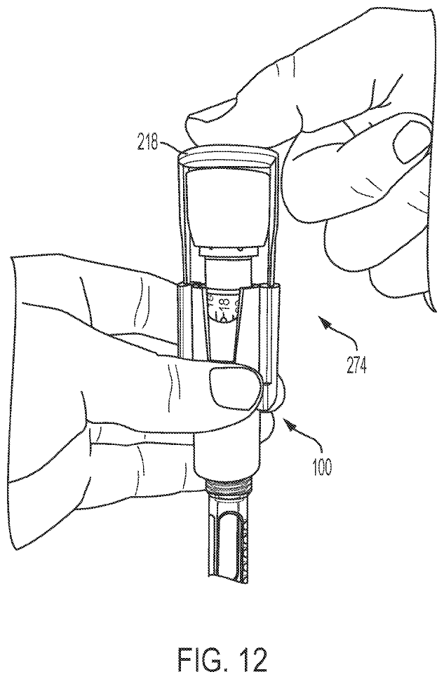

FIGS. 11A-11B and FIG. 12 illustrate a user's grip facilitated by stabilizing apparatus 200 (equally applicable to stabilizing apparatus 1200, 2200, 3200, 4200, 5200, 7200) after the dose has been set. The components and coupling therebetween allow for several varied grips, including those illustrated by FIGS. 11A and 11B, to allow the user to hold and utilize device 100 (equally applicable to devices 1100, 2100, 3100) in a manner that provides most comfort to the user while allowing for effective use of device 100 without interference with movement of various translatable or rotatable components of the device and to facilitate usage of the device by users with dexterity impairment. Grip 268 in FIG. 11A illustrates use of the medical delivery device 100 by holding the medical delivery device 100 with all of the user's fingers positioned on one side of the medical delivery device 100 and positioning the user's thumb at an outer edge 270 of slide button 218 adjacent to rail 210 to apply the requisite force F1 to slide button 218. Grip 272 in FIG. 11B illustrates use of the medical delivery device 100 in a syringe-type technique as described above, wherein a user may position a finger on each ledge 214 of guide body 202 while positioning user's thumb on slide button 218 to apply the requisite force to slide button 218. FIG. 12 illustrates an example of a two-handed grip 274 with the holding of the medical delivery device 100 with one hand, shown gripping the guide body and/or the body of the medication delivery device, and the user's other hand applying the requisite force to slide button 218 and deliver medication. Although the user's thumb is shown applying the force F1, the user's palm or one of the user's other fingers may apply the force.

Once an appropriate grip is taken and the dose has been set, the user may dispense the dose. Referring to FIGS. 1-2, an axial distal force F1 is applied to the proximal shielding surface 220 of the slide button 218 by the user to initiate dose dispensing. Such distal force F1 is transmitted to the actuator. For example, when no data collection device is attached (see, for example, FIG. 8 and FIG. 13), such force is transmitted through the slide button, directly to the actuator or activation button of the actuator. In another example, when a data collection device is attached (see, for example, FIG. 2 and FIG. 14), such force is transmitted through the slide button and directly to an input element (if present) of data collection device or upper housing of data collection device (if so configured) to allow relative movement between parts of the housing and then to the actuator or activation button of the actuator. Regardless of configuration, the actuator of the device 100 is initiated, and rotation of the actuator and/or data collection device in the dose dispensing direction, opposite the dose setting direction, causes screwing of the dose setting member 17 into the device toward the zero position, and distal travel of the actuator and/or data collection device relative to device 100. Consequently, the shielding surface 220 of the slide button 218 moves distally toward the guide body 202 that is coupled to device 100, as the rail 210 slides further into the receiver 206 of the guide body 202. As such, the shielding surface of the stabilizing apparatus provides the benefit of shielding the axial force provided by the user from affecting rotation of the actuator and/or data collection device, and, in some embodiments, from also affecting the radial outer surface of the rotating actuator and/or data collection device. The stabilizing apparatuses provided herein may also provide the benefit of focusing the input force F1 closer to, if not directly along, the central axis AA of the device 100. The rail system of the stabilizing apparatus may also provide the benefit of distributing forces more evenly across the slide button to facilitate movement during dose dispensing. Additionally, referring to embodiments illustrated in FIGS. 6-7, some embodiments of data collection devices may not require relative motion between the tab 1250 and the device 1100 during dose dispensing, which may provide the benefit of preventing. undesirable rotation of the data collection device tab during dosing.

Similar to FIG. 6 wherein the slide button is shown to engage directly with the actuator, FIG. 13 shows another embodiment of the stabilizing apparatus 4200. The guide body 4202 is shown formed integrally with the proximal portion of the body of device. Although the guide body 4202 may be slid onto the device 4100, as earlier, guide body 4202 is shown here to be molded integrally with the body of the device 4100. Any embodiment of guide bodies described herein may be integrally formed with the device body. Various embodiments of the slide button 4218 described herein can be molded as a single piece injection molded plastic.

FIG. 14 shows another embodiment of the stabilizing apparatus 5200 with the slide button 5212 that couples directly with the actuator (or as shown directly with the data collection device 5226). In other words, the data collection device 5226 does not have the tab. The slide button 5212 includes the shielding surface 5220 in form a disk body of rigid material, such as medical grade polymer. The disk body may include a pair of oppositely facing radial protrusions 5222 that couple to corresponding rails 5210. The rails 5210 may have a high strength to size ratio to facilitate a smaller cross-sectional area size. In one example, the rails 5210 are made from a medical grade metal, such as stainless steel. Although the guide body 5202 may be slid onto the device body, as described above, guide body is shown here to be molded integrally with the body of device 5100. Because of the smaller size of the rails 5210, the receiver 5206 of the guide body 5202 may be sized correspondingly to reduce the overall cross-sectional area of the guide body 5202 and the body of device 5100 if integrally formed.

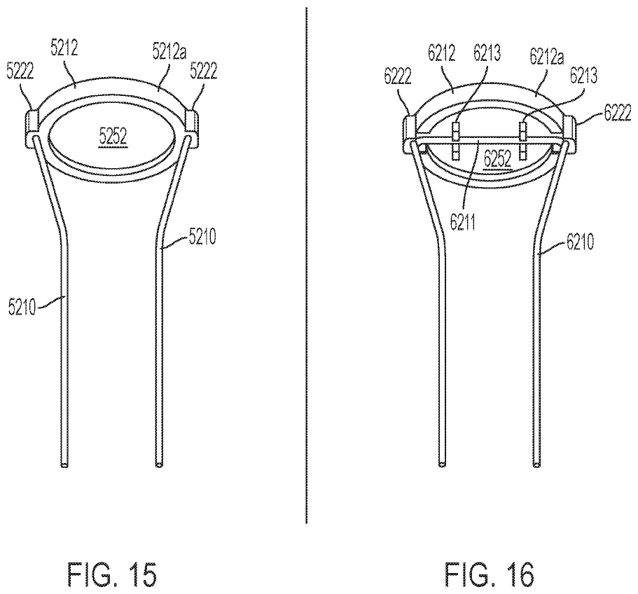

FIG. 15 shows one embodiment of the slide button 5212 in FIG. 14 useful in the stabilizing apparatus 5200, showing the proximal ends of the rails 5210 fixedly secured to corresponding radial protrusions 5222. The rails 5210 may be linear or may be shaped to correspond to the outer pen body. Rails 5210 are shown extending radially inward gradually in the distal direction along the longitudinal axis AA to a point where the distal portion of the rails 5210 are in parallel (within acceptable tolerances) to one another and to the longitudinal axis. Attachment element 5252 is shown on the distal end of the disk body 5212a of the slide button 5212. Here, the attachment element 5252 comprises a recess sized and shaped to couple to at least the proximal end of the data collection device 5226 via fit or to the proximal end of the actuator. Attachment element 5252 may be configured to allow rotation of the data collection device 5226 relative to the slide button 5212.

FIG. 16 shows another embodiment of the slide button 6212 in FIG. 14, showing the rails 6210 as a single piece being fixedly secured to disk body 6212a of the slide button 6212. Rails 6210 can have multiple bends. Rails 6210 are shown extending from a proximal connecting segment 6211 radially inwardly gradually in the distal direction along the longitudinal axis AA to a point where the distal portion of the rails 6210 are in parallel. The proximal connecting segment 6211 may extend orthogonal to the longitudinal axis AA and span across the distal end of the disk body 6212a. Proximal connected segment 6211 may be securely fixed to the distal end of the disk body 6212a. Distal end of the disk body 6212a is shown having interference ribs 6213 to receive and retain the proximal connecting segment 6211 that spans across the disk body. Alternatively, the proximal connecting segment 6211 may be attached to the distal end of disk body 6212a by other attachment means, such as heat staking, ultrasonic staking, snap fit, etc. Attachment element 6252 is shown as a recess, similar to the recess in attachment element 5252. The depth of the recess is sized to allow for the distal extension of the interference ribs 6213 and still receive a sufficient portion of the data collection device or actuator.

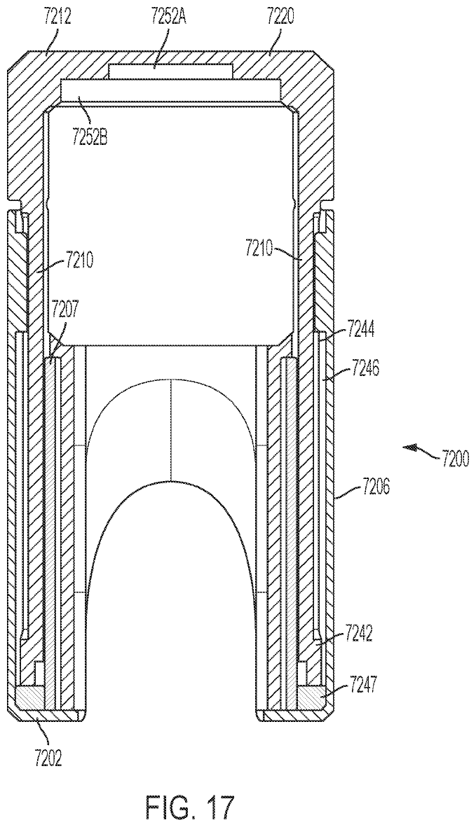

FIG. 17 shows another embodiment of the stabilizing apparatus 7200, with the guide body 7202 and the slide button 7212 shown in the fully inserted position. The disk body of the shielding surface 7220 is shown with the attachment elements 7252A, B sized and shaped for the tab of the data collection device (not shown) and the proximal end of the data collection device, respectively. Each rail 7210 is shown including the limit element 7242, which is configured to engage with the lip 7244 within pocket 7246 of receiver 7206 of the guide body 7202. The receiver 7206 may define a proximal edge 7207 that is slidably engageable along the distal end surface of the data collection device. A bumper 7247 may be included inside the pocket 7246. Bumper 7247 may be disposed at the bottom of the pocket and may be formed from a soft durometer material, such as an elastomer, to cushion the impact of the end of the rails when traveling to the position shown in FIG. 17.

The dose detection device described herein uses a sensing component and a sensed component. One of these components may be coupled (directly or indirectly) to members of the medication delivery device. Various sensor systems are contemplated herein. The term “sensing component” refers to any component which is able to detect the relative position of the sensed component. The sensing component includes a sensing element, or “sensor”, along with associated electrical components to operate the sensing element. The “sensed component” is any component for which the sensing component is able to detect the position and/or movement of the sensed component relative to the sensing component. For the dose delivery detection device, one of the sensed component or the sensing component rotates relative to the other, which is able to detect the angular position and/or the rotational movement of the rotating sensed component or sensing component. The sensing component may comprise one or more sensing elements, and the sensed component may comprise one or more sensed elements. The sensor system is able to detect the position or movement of the sensed component(s) and to provide outputs representative of the position(s) or movement(s) of the sensed component(s). Sensing and determining data may occur prior to dose setting, during dose setting, during dose delivery, or after dose delivery. Information may include time/date, dose set amount, dose delivered amount, product identification data, battery life remaining, errors codes, as well as other information about the operation of the device.

A sensor system typically detects a characteristic of a sensed parameter which varies in relationship to the position of the one or more sensed elements within a sensed area. The sensed elements extend into or otherwise influence the sensed area in a manner that directly or indirectly affects the characteristic of the sensed parameter. The relative positions of the sensor and the sensed element affect the characteristics of the sensed parameter, allowing the controller of the sensor system to determine different positions of the sensed element. Suitable sensor systems may include the combination of an active component and a passive component. With the sensing component operating as the active component, it is not necessary to have both components connected with other system elements such as a power supply or controller. The data collection device housing may include a first part that rotates relative to a second part. One of the first or second parts of the dose detection device is associated with a sensed component, and the other of the first or second parts is associated with a sensing component. Sensing of relative rotation may occur during dose setting and/or dose delivery. In one embodiment, the housing of the data collection device may include two housing components that rotate together during dose setting and that rotate relative to one another during dose delivery. In another example, the two housing components may be axially movable relative to one another such that the upper part is movable down relative to the lower part to allow activation of the device. In another example, a portion of the housing of the data collection device may be counted as the first part, and an axially movable button on top of the device, such as the tab described herein, may constitute the second part.

Any one of a variety of sensing technologies may be incorporated by which the relative positions of two members can be detected. Such technologies may include, for example, technologies based on tactile, optical, magnetic, acoustical, inductive or electrical measurements.

In one aspect, the sensor system detects relative positions or movements of the rotating sensed elements or sensing elements, and therefore of the associated members of the medication delivery device. The sensor system produces outputs representative of the position(s) or the amount such movement. For example, the sensor system may be operable to generate outputs by which the rotation of the rotating dose member during dose delivery can be determined. A controller is operably connected to sensor(s) to receive the outputs. In one aspect, the controller may be configured to determine from the outputs the amount of dose delivered by operation of the medication delivery device. In another aspect, the controller may be configured to determine from the outputs data that may be used to determine the amount of dose delivered by operation of the medication delivery device.

With the extent of rotation having a known relationship to the amount of a delivered dose, the sensor system operates to detect the amount of angular movement from the start of a dose injection to the end of the dose injection. For example, a typical relationship for a pen injector is that an angular displacement of a rotating dose member of 18° is the equivalent of one unit of dose, although other angular relationships are also suitable. The sensor system is operable to determine the total angular displacement of a rotating dose member during dose delivery. Thus, if the angular displacement is 90°, then 5 units of dose have been delivered. One approach for detecting the angular displacement is to count increments of dose amounts as the injection proceeds. For example, a sensor system may use a repeating pattern of sensed elements, such that each repetition is an indication of a predetermined degree of angular rotation. Conveniently, the pattern may be established such that each repetition corresponds to the minimum increment of dose that can be set with the medication delivery device.

An alternative approach is to detect the start and stop positions of the relatively moving member, and to determine the amount of delivered dose as the difference between those positions. In this approach, it may be a part of the determination that the sensor system detects the number of full rotations of the rotating dose member. Various methods for this are well within the ordinary skill in the art and may include “counting” the number of increments to assess the number of full rotations.

The dose detection device described herein may be permanently integrated with the medication delivery device or removably attached to the medication delivery device. In an illustrative embodiment, as least some of the dose detection device components are provided in the form of a module that is removably attached to the medication delivery device. This has the advantage of making these sensor components available for use on more than one pen injector.

While this invention has been described as having exemplary designs, the present invention can be further modified within the spirit and scope of this disclosure. This application is therefore intended to cover any variations, uses, or adaptations of the invention using its general principles. Further, this application is intended to cover such departures from the present disclosure as come within known or customary practice in the art to which this invention pertains and which fall within the limits of the appended claims.

Various aspects are described in this disclosure, which include, but are not limited to, the following aspects:

1. An apparatus for a medication delivery device, the medication delivery device having a body, and a user dose setter coupled to a proximal end of the body. The user dose setter including a proximal surface facing away from a needle end of the body and a circumferential surface extending from the proximal surface and defined about a longitudinal axis of the medication delivery device. The apparatus including a guide body associated with the medication delivery device, a slide button axially movable relative to the guide body, and at least one rail coupled between the guide body and the slide button. The slide button includes a shielding surface and a shielding lip. The shielding surface at least partially covers the proximal surface of the user dose setter of the medication delivery device. The shielding lip extends toward the needle end along the circumferential surface of the user dose setter of the medication delivery device. During dose setting and delivery, the slide button is rotationally free relative to the user dose setter and axially movable relative to the guide body. During dose setting, the user dose setter is rotated in a first direction, the user dose setter together with the slide button proximally move away from the body of the medication delivery device from a first position to a second position.

2. The apparatus of aspect 1, wherein the at least one rail comprises a plurality of rails, wherein at least one of the plurality of rails define said shielding lip, each of the rails circumferentially spaced from one another to define windows therebetween, the windows sized to allow access for rotation of the user dose setter during dose setting.

3. The apparatus of aspect 2, wherein the slide button comprises a distal surface comprising a protrusion to transmit an axial force to the user dose setter of the medication delivery device during dose delivery, wherein during dose delivery, the user dose setter together with the slide button distally move toward the body of the medication delivery device to the first position.

4. The apparatus of any one of aspects 1-3, wherein the user dose setter of said medication delivery device comprises an actuator, the slide button engageable with a proximal surface of the actuator.

5. The apparatus of aspect 4, wherein the user dose setter comprise a data collection device coupled over the actuator, wherein the data collection device is axially fixed relative to the slide button prior to engagement to the data collection device.

6. The apparatus of one of aspects 4-5, wherein the at least one rail slide relative to the guide body, having a range of translational motion sufficient to couple the guide body to the slide button when the medication delivery device is fully extended, to deliver maximum dose, or fully depressed, after administering a complete dose.

7. The apparatus of any one of aspects 1-6, wherein the at least one rail has a limit element to prohibit axial movement of the slide button during dose setting beyond a predefined axial distance away from the body of the medication delivery device when associated with the medication delivery device.

8. The apparatus of any one of aspects 1-7, wherein the slide button comprises a distal surface comprising an attachment element configured to couple to a portion of the user dose setter.

9. The apparatus of aspect 8, wherein the user dose setter includes an input button to facilitate initiation of the dose delivery, and the attachment feature couples to the input button.

10. The apparatus of any one of aspects 1-9, wherein the guide body is removably attached to the body of the medication delivery device.

11. The apparatus of aspect 10, wherein the guide body includes a recess to receive a protrusion of the body of the medication delivery device for attachment between the guide body and the body of the medication delivery device.

12. A medication delivery system including a medication delivery device disposed about a longitudinal axis and having an actuator, and a data collection device couplable to the actuator and rotationally and axially fixed relative to the actuator. The data collection device includes an input element that is axially movable relative to the data collection device and is configured to activate the actuator for dose delivery. A guide body is associated with the medication delivery device. A slide button is axially movable relative to the guide body and rotationally free relative to the data collection device. The slide button includes an attachment element configured for removable attachment to a portion of the data collection device. A first rail and a second rail are coupled between the guide body and the slide button. The slide button includes a shielding surface at least partially covering a proximal surface of the data collection device. The first and second rails and the shielding surface are arranged to provide a window to define a human graspable section of the data collection device for rotation of the data collection device for dose setting of the medication delivery device.

13. The medication delivery system of aspect 12, wherein the shielding surface covers at least 50% of the proximal surface of the activation button.

14. The medication delivery system of aspect 13, wherein the shielding surface covers the entirety of the proximal surface of the activation button.

15. The medication delivery system of any one of aspects 12-14, wherein the attachment element comprises a cylindrical wall extending about the longitudinal axis of the medication delivery device from a distal surface of the slide button and is engaged to a radially outer surface of the portion of the data collection device.

16. The medication delivery system of any one of aspects 12-15, wherein the first and second rails are coupled to the slide button and received by a receiver of the guide body.

17. The medication delivery system of any one of aspects 12-16, wherein the guide body further includes a cover for a bezel of the medication delivery device.

18. The medication delivery system of any one of aspect 12-17, wherein the guide body is formed integrally with a body of the medication delivery device.

19. The medication delivery system of any one of aspects 12-18, wherein the first and second rails comprises a metal material.

20. The medication delivery system of any one of aspects 12-19, wherein the medication delivery device includes a reservoir of medication.

21. An assembly for a medication delivery device including a guide body having at least one receiver, at least one rail positionable within the at least one receiver, a slide button coupled to the at least one rail opposite the at least one receiver, and a data collection device positioned intermediate of the slide button and the guide body, the data collection device configured to facilitate coupling of the stabilizing apparatus to the medication delivery device.

22. The assembly of aspect 19, wherein the slide button includes a distal surface including an attachment element configured to couple to a portion of the data collection device.

23. A method of removably attaching a stabilizing apparatus to a medication delivery device, the method including or more of the following steps: providing an apparatus comprising a guide body having at least one receiver, a slide button having at least one rail positioned within the at least one receiver, and a data collection device coupled to the slide button such that the data collection device is positioned intermediate of the slide button and the guide body; providing a medication delivery device; inserting the medication delivery device within a through hole defined by the guide body; and attaching an actuator of the medication delivery device to the data collection device.

24. The method of aspect 23, wherein a distal surface of the slide button includes an attachment element, wherein the attachment element is coupled to a portion of data collection device such that the data collection device is axially fixed relative to the slide button to define an assembly.

25. The method of aspect 23, further comprising coupling the data collection device to the slide button after the attaching step.

26. The method of aspect 23, further comprising coupling the data collection device to the slide button prior to the inserting step.

26. The method of any one of aspects 23-26, further including: after a use of the medication delivery device, detaching the actuator of the used medication delivery device from the data collection device; and removing the medication delivery device from the though hole of the guide body.

1. An apparatus for a medication delivery device, the medication delivery device having a body, a user dose setter coupled to a proximal end of the body, the user dose setter comprising a proximal surface facing away from a needle end of the body and a circumferential surface extending from the proximal surface and defined about a longitudinal axis of the medication delivery device, the apparatus comprising:
a guide body configured to be associated with the medication delivery device, the guide body having a pair of receivers, each of the receivers having a pocket extending longitudinally along the longitudinal axis;
a slide button axially movable relative to the guide body;
a pair of rails coupled between the guide body and the slide button, each of the rails circumferentially spaced from one another to define a pair of opposite-facing windows therebetween, each of the opposite-facing windows sized to allow access for rotation of the user dose setter during dose setting, each of the rails slidably coupled within the pocket of a corresponding one of the receivers;
wherein the slide button includes a shielding surface, the shielding surface at least partially covering the proximal surface of the user dose setter of the medication delivery device, wherein during dose setting and delivery the slide button is rotationally free relative to the user dose setter and axially movable relative to the guide body;
wherein during dose setting the user dose setter is rotated in a first direction, the user dose setter together with the slide button proximally move away from the body of the medication delivery device from a first position to a second position.

2. (canceled)

3. The apparatus of claim 1, wherein the slide button comprises a distal surface comprising a protrusion to transmit an axial force to the user dose setter of the medication delivery device during dose delivery, wherein during dose delivery, the user dose setter together with the slide button distally move toward the body of the medication delivery device to the first position.

4. The apparatus of claim 1, wherein the user dose setter of said medication delivery device comprises an actuator, the slide button engageable with a proximal surface of the actuator.

5. The apparatus of claim 4, wherein the user dose setter comprise a data collection device coupled over the actuator, wherein the data collection device is axially fixed relative to the slide button prior to engagement to the data collection device.

6. The apparatus of claim 1, wherein the each of the rails slide relative to the guide body, having a range of translational motion sufficient to couple the guide body to the slide button when the medication delivery device is fully extended, to deliver maximum dose, or fully depressed, after administering a complete dose.

7. The apparatus of claim 1, wherein the at least one rail has a limit element to prohibit axial movement of the slide button during dose setting beyond a predefined axial distance away from the body of the medication delivery device when associated with the medication delivery device.

8. The apparatus of claim 1, wherein the slide button comprises a distal surface comprising an attachment element configured to couple to a portion of the user dose setter.

9. The apparatus of claim 8, wherein the user dose setter includes an input button to facilitate initiation of the dose delivery, and the attachment feature couples to the input button.

10. The apparatus of claim 1, wherein the guide body is removably attached to the body of the medication delivery device.

11. The apparatus of claim 10, wherein the guide body includes a recess to receive a protrusion of the body of the medication delivery device for attachment between the guide body and the body of the medication delivery device.

12. A medication delivery system, comprising;
a medication delivery device disposed about a longitudinal axis, having an actuator;
a data collection device couplable to the actuator and rotationally and axially fixed relative to the actuator, the data collection device including an input element that is axially movable relative to the data collection device and configured to activate the actuator for dose delivery;
a guide body associated with the medication delivery device, the guide body having a pair of receivers, each of the receivers having a pocket extending longitudinally along the longitudinal axis;
a slide button axially movable relative to the guide body and rotationally free relative to the data collection device, wherein the slide button includes an attachment element configured for removable attachment to a portion of the data collection device;
a first rail and a second rail coupled between the guide body and the slide button;
wherein the slide button includes a shielding surface at least partially covering a proximal surface of the data collection device, wherein the first and second rails and the shielding surface are arranged to provide a window to define a human graspable section of the data collection device for rotation of the data collection device for dose setting of the medication delivery device.

13. The medication delivery system of claim 12, wherein the shielding surface covers at least 50% of the proximal surface of the activation button.

14. The medication delivery system of claim 13, wherein the shielding surface covers the entirety of the proximal surface of the activation button.

15. The medication delivery system of claim 12, wherein the attachment element comprises a cylindrical wall extending about the longitudinal axis of the medication delivery device from a distal surface of the slide button and is engaged to a radially outer surface of the portion of the data collection device.

16. The medication delivery system of claim 12, wherein the guide body comprise a pair of receivers, and the first and second rails are coupled to the slide button and received by the corresponding receiver of the guide body.

17. The medication delivery system of claim 12, wherein the guide body further comprises a cover for a bezel of the medication delivery device.

18. The medication delivery system of claim 12, wherein the guide body is formed integrally with a body of the medication delivery device.

19. The medication delivery system of claim 12, wherein the first and second rails comprises a metal material.

20. The medication delivery system of claim 12, wherein the medication delivery device includes a reservoir of medication.

21-27. (canceled)

28. A medication delivery system, comprising;
a medication delivery device disposed about a longitudinal axis, having an actuator;
a data collection device couplable to the actuator and rotationally and axially fixed relative to the actuator, the data collection device including an input element that is axially movable relative to the data collection device and configured to activate the actuator for dose delivery;
a guide body associated with the medication delivery device, the guide body having a pair of receivers, each of the receivers having a pocket extending longitudinally along the longitudinal axis, the pocket of each of the receivers having a lip, and a bumper at a bottom of the pocket;
a slide button axially movable relative to the guide body and rotationally free relative to the data collection device, wherein the slide button includes a shielding surface at least partially covering a proximal surface of the data collection device;
a first rail and a second rail coupled between the guide body and the slide button, each of the first and second rails slidably coupled within the pocket of a corresponding one of the receivers, each of the first rail and the second rail including a limit element configured to engage with the lip within the pocket of the corresponding one of the receivers;
wherein the first and second rails and the shielding surface are arranged to provide a window to define a human graspable section of the data collection device for rotation of the data collection device for dose setting of the medication delivery device.
